(19) United States
(12) Patent Application Publication
(10) Pub. No.: US 2006/0287682 A1
(43) Pub. Date: Dec. 21, 2006

(54) Multi-site pacemaker with slaved eletrodes network

(76) Inventors: Josy Cohen, La Plesns-Trevise (FR); Francis Joffre, Bures sur Yvette (FR); Maurice Salichon, Gif Sur Yvette (FR); Nicolas Bonnet, Paris (FR); Mourad Karouia, Bagneux (FR)

Correspondence Address: Robert E. Krebs;Thelen Reid & Priest LLP, P.O. Box 640640, San Jose, CA, 95164-0640, US

(21) Appl. No.: 11/176,499

(22) Filed: Jul. 6, 2005

(30) Foreign Application Priority Data
Jul. 7, 2004 (FR) 04 07567

(51) Int. Cl. A61N 1/36

(52) U.S. Cl. 607/9

(57) ABSTRACT

The invention relates to an implantable pacemaker. This pacemaker comprises at least one conducting cable (11) electrically connecting a control box (10) to at least one electrode placed at a point in the patient's heart, the said control box (10) comprising a power supply system and an electronic system, and is characterised in that at least part of the said electronic system is remote from the control box (10) and is located at the at least one electrode to form an electrode module (13, 14a, 14b), the electrode module being placed on the outside surface of the heart.

### TECHNICAL DOMAIN

The invention relates to a pacemaker capable of pericardial (on its outside face) or endocavitary (inside cardiac cavities) stimulating and/or exploring the heart using electrodes at judicially chosen locations in the heart. In particular, the invention relates to a multi-site pacemaker comprising slaved electrodes arranged on the meshes of a network and in which each site is fitted with an electrode to stimulate and/or explore the heart.

This device is intended particularly for use by patients with heart failures.

### STATE OF PRIOR ART

The heart is an organ composed of muscular fibres or cells that in particular propagate electric signals that provoke contraction of the atriums and ventricles of the heart as they propagate. The electrical signal is created at a precise point of the heart, the sinoatrial node, located in the right atrium. The sinoatrial node regularly emits weak electrical pulses that are transmitted by a relay, the atrioventricular node, to the cardiac muscular fibres. To obtain a contraction, the cardiac muscular cells depolarise electrically, thus creating a depolarisation wave that step by step causes electrical depolarisation of all muscular cells of the heart. At a determined time after this wave has passed, a mechanical contraction wave propagates in the atriums and the ventricles, thus contributing to pumping blood in and outside the heart. FIG. 1 shows a diagram of a heart 1 comprising a right atrium .2 and a left atrium 3 in the top part of the heart, a right ventricle 4 and a left ventricle 5 in the bottom part. Blood arrives in the right atrium 2 through the vena cava 6, passes into the right ventricle 4 and then goes into the lungs through the pulmonary trunk 7. Blood then returns through the pulmonary veins 8 and enters the left atrium 3, then the left ventricle 5 and exits through the aorta 9. When the heart function is defective, it can be stimulated by creating a new electrical depolarisation wave or by restarting a wave that was not completed at a given point of the heart. This cardiac stimulation is usually achieved by using an apparatus called a pacemaker that is implanted in the patient's body close to the heart.

At the present time, implantable pacemakers are composed of a control box, at least one conducting cable and at least one electrode. The control box comprises a power supply system (a battery) capable of outputting electrical pulses at regular intervals and a more or less complex control electronics that controls intervals between the output electrical pulses. These electrical pulses are transmitted to the heart through the conducting cable called the electrode cable, that sets up electrical contact between the box and the heart. An electrode is put into place at the end of the cable and is brought into contact with the heart. Several electrodes can be arranged at the end of the same cable. Similarly, the pacemaker may be provided with several cables. Some of these electrodes may perform a stimulation function, and others an exploration function of the electrical state of the heart. They may also perform these two functions simultaneously.

The problem with existing pacemakers is that their control box has to be changed about every 4 years due to wear in the power supply system. A surgical operation is necessary every time that the system is replaced, with all the attendant risks for example such as risks of infection, nosocomial diseases, or risks related to the surgical operation itself.

Moreover, existing pacemaker electrodes must be placed on the heart individually. The practitioner must put them into place one by one, inserting them into the endocavitary of the heart through the veins, observe their effect and possibly reposition them so as to improve and optimise function of the heart. Therefore positioning the electrodes on the heart is a difficult and tedious step.

Another problem is the size of the electrode cables. Electrode cables and electrodes are usually routed to the heart by inserting them in veins or arteries leading to the heart (endocavitary path). Document [1] describes an example of such a pacemaker. In such a pacemaker, the number of electrode cables is limited by the diameter of the veins or arteries and by the size of electrode cables. At the present time, pacemakers with a maximum of three electrode cables are available on the market. It is easy to understand that with existing pacemakers, increasing the number of stimulation sites will be hampered by a technological limit due to anatomophysiological impossibilities, if only due to the steric size of the electrode cables.

This problem can be solved by making electrode cables with branch connections. Documents [1] and [2] describe examples of pacemakers using this type of electrode cables with branch connections. The disadvantage of this solution is that the use of branch connections in the electrode cables increases the electrical resistance of the cables and causes a voltage drop. However, a sufficient stimulation must be initiated at a point in the heart under specific minimum electrical conditions (voltage, power or energy) to propagate an electrical depolarisation wave in the cardiac muscular cells. With this system, the electrical power necessary to uniformly stimulate each stimulation site cannot be supplied by the battery normally used in the pacemakers technology. The use of electrode cables with branch connections is only possible if a power supply system with a greater capacity and a higher output voltage, and consequently a larger and heavier system, is used.

### PRESENTATION OF THE INVENTION

The purpose of the invention is to supply a pacemaker that can be implanted in a patient's body and that does not have the disadvantages of prior art. This purpose is achieved using an implantable pacemaker comprising at least one conducting cable electrically connecting a control box to at least one electrode placed at a point in the patient's heart, the control box comprising a power supply system and an electronic system, characterised in that at least one part of the electronic system is remote from the control box and is located at the at least one electrode to form an electrode module, the electrode module being placed on the surface of the heart.

According to one variant, at least part of the power supply system is remote from the control box and is located at at least one electrode module.

Advantageously, the remote part of the electronic system consists of processing and/or control electronics used by the module to explore and/or stimulate the heart.

Therefore the electrode modules are composed of processing and/or control electronics. These electrode modules are placed at judiciously chosen positions on the heart.

The processing electronics processes a signal, while the control electronics applies control over triggering a stimulation.

If the electrode module only comprises processing electronics, then it can only operate in exploration mode, in other words to study the behaviour of the heart.

If the electrode module only comprises control electronics, then it can only operate in stimulation mode.

If the pacemaker only comprises the electrode module in stimulation mode, then all heart stimulations take place at regular intervals regardless of the state of the heart, in other words regardless of whether or not the heart needs an artificial stimulation.

If the pacemaker comprises electrode modules in exploration mode and in stimulation mode, it is useful to slave the electrode modules in stimulation mode to the electrode modules in exploration mode. In other words, the electrode modules in stimulation mode are made to be dependent on information output by the electrode modules in exploration mode. The result is thus a means of regulating the stimulation rate applied by modules in stimulation mode to the heart rate necessary to compensate for the heart failure of the patient wearing the stimulator. Thus, the pacemaker only sends stimulations when the heart needs them. The stimulator adapts to the patient's lifestyle.

Finally, each electrode module may comprise processing electronics and control electronics. In this case, each electrode module can be changed over to be configured in exploration mode or in stimulation mode, depending on the needs. In this case, the electrode module can process all signals from the heart, it can process all signals from other modules and apply control over triggering a stimulation. In general, the electrode module will be in exploration mode most of the time and will change over to stimulation mode only if it perceives the need for stimulation of the heart.

Advantageously, the processing electronics that the electrode module uses to explore the heart comprises at least one sensor that can be chosen from among the following sensors:
one or more accelerometers that can adapt the heart rate to suit the lifestyle of the patient wearing it;
one or more accelerometers used to measure the contractibility of the myocardium (contraction of the equipped ventricles and atriums),
one or more respiration sensors (movement of the thoracic cage) so that an accelerometer can better adapt the heart rate to the patient's lifestyle,
one or more movement sensors capable of performing the same functions as the accelerometer,
one or more piezo-electric sensors capable of acquiring information on the heart activity,
one or more blood temperature sensors,
one or more blood oxygenation sensors,
one or more blood pressure sensors,
one or more flowmeters for measuring the blood flow,
one or more probes for picking up the myocardium depolarisation wave. . . .

Advantageously, the processing electronics enabling the electrode module to explore the heart comprises a means of selecting signals with a determined frequency. This means is advantageously a filter.

The processing electronics, and particularly the filter, is either placed in the electrode module or in the control box, depending on whether the electronic system is partially or totally remote.

Advantageously, the control electronics enabling the electrode module to stimulate the heart comprises a means capable of outputting an electrical stimulation to the heart. This means is advantageously a capacitor.

In the example in FIG. 3, the capacitor is inside the processing electronics. The capacitor may be in the electrode module or in the control box depending on the degree of delocalisation.

Advantageously, the electronic system located in the electrode modules is miniaturised Advantageously, the part of the remote electronic system is a system on a chip. A <<system on chip>> (SoC) is preferably designed using microtechnology. For example, the latest developments in microtechnology (ASIC, FPGA, MENS, . . . ) will be used.

In one particular case, the pacemaker comprises at least two electrode modules connected to each other by a means that surrounds at least part of the heart.

This means is put into place on all or on only part of the heart. It may be necessary to stimulate only a particular part of the heart, for example if the other parts of the heart are composed of healthy tissues.

Advantageously, the means that surrounds all or part of the heart is used for the mechanical support of the at least two electrode modules.

Advantageously, the means provides contention for the part of the heart surrounded by it. This is particularly useful when treating a patient suffering from ventricular hypertrophy.

Advantageously, the means is a means for electrical communication between the electrode modules.

Advantageously, the means is a means for distributing energy between the electrode modules.

The means can thus provide mechanical support for electrode modules on the heart, provide contention of the heart, carry communications between electrode modules, and/or distribute energy.

Advantageously, if the means act at least as an energy distributor, then this means or an electrode module located on this means can be connected to the control box through a conducting cable. In this special case, there is only one conducting cable supplying electrical power supply to all electrode modules located on this means.

The parts from which the pacemaker is made that come into contact with the heart and its environment are preferably made of biocompatible materials. Moreover, the component parts of the pacemaker that come into contact with the heart and its environment, except for the electrodes, are made from an insulating material. For example, these parts may be coated with silicone or titanium.

Advantageously, the means used for communication between the electrode modules and/or the energy distribution is a multi-wire coaxial cable.

Advantageously, the means used for mechanical support of the electrode cables on the heart, contention of the heart, communication between the electrode modules and/or energy distribution is a net comprising a mesh. For example, it may consist of a set of interlaced meshes. The electrode modules are placed on the meshes of the net at judiciously chosen sites that may or may not be located at nodes (intersection) of the meshes. The mesh of the net may or may not be regular, and may or may not be dense.

Electrode modules are organised in a network in the form of a mesh, a net, a cluster, a tree structure or any other geometry adapted to a cardiac application that can stimulate the heart with just the right amount of energy and in stimulation space-time pattern providing the best coordinated contraction of each of the cardiac cavities during a cycle.

Advantageously, the mesh of the net is made from multi-wire and coaxial helical conductors covered by an electrically insulating material biocompatible with the human tissues with which they are in contact. This is particularly practical when the electrode modules comprise processing electronics and also control electronics. The presence of multi-wire conductors enables electrode modules to operate in bipole mode in a closed electrical circuit, in other words in exploration mode or in stimulation mode; electrical energy is carried to the electrodes to stimulate or explore the myocardium through a closed electrical circuit.

According to one particular embodiment, the implantable pacemaker according to the invention also comprises a communication means for the remote configuration of the electrode modules, the said communication means being located in the control box. The practitioner can thus modify operation of the electrode modules without needing to operate the patient to access the control box and the electrode modules, for example by changing over a determined electrode module to act in exploration mode rather than in stimulation mode.

Advantageously, this communication means comprises an antenna and operates by remote transmission.

Advantageously, only the antenna of the communication means is located in the control box, the rest of the elements making up the communication means being localised on the means for connecting the electrode modules.

The electrode modules may comprise only one control electronics, or they may be electrode modules acting as control electronics and also processing electronics that are switched into stimulation mode.

Advantageously, at least one electrode module is configured in exploration mode and transmits information about the state of the heart to the other electrode modules. In this case, the electrode module in question may comprise only one processing electronics, or it may comprise both control electronics and processing electronics and be switched in exploration mode. The same electrode module can act for exploration and as a stimulator.

Advantageously, at least one electrode module is configured in exploration mode and in stimulation mode.

Advantageously, all electrode modules are located on the means of connecting the electrode modules to each other.

Advantageously, at least one electrode module configured in exploration mode is not located on the means of connecting the electrode modules together, but is electrically connected to it. It can be decided to position the electrode module configured in exploration mode on the means of connecting the electrode modules to each other or to locate it outside the said means and to electrically connect the module to the means.

Advantageously, the electrode module configured in exploration mode is capable of executing is the following sequence of steps:
a)—analyse the electrical state of the heart at the location of the electrode module configured in exploration mode,
b)—when receiving an electrical signal originating from the heart, calculate the frequency of the said signal to determine whether or not the heart is in a depolarised or a repolarised state,
c)—if the heart is in a depolarised state, then send a frequency encoded signal to the means of exchanging information between electrode modules, the frequency coding giving information about the time separating two repolarisation states of the heart.

Advantageously, the pacemaker comprises at least one electrode module, configured in stimulation mode, which can execute the following sequence of steps:
a)—wait for a frequency encoded signal originating from a module in exploration mode,
b)—after detection of the encoded signal, start the internal clock and wait for a determined set time,
c)—analyse the electrical state of the heart at the location of the electrode module in stimulation mode,
d)—if the electrode module configured in stimulation mode receives an electric signal from the heart with a value above a determined threshold, then the internal clock is stopped and the set time is reset to zero,
e)—if the electrode module in stimulation mode does not receive an electric signal from the heart, then the module outputs an electrical stimulation at the position at which it is located on the heart, the internal clock is stopped and the set time is reset to zero.

The electrode module in exploration mode monitors the frequency and possibly the amplitude of heart pulses, and sends a stimulation order to electrode modules in stimulation mode to notify them that the time between two successive stimulations has to be increased or reduced. This regulates the stimulation rate so that the heart rate necessary to compensate for the heart failure of the patient wearing the pacemaker can be maintained. The pacemaker thus adapts itself to the patient's lifestyle (fast walking, rest, etc.). Furthermore, as a result of the monitoring done by the electrode module(s) in exploration mode, the stimulation is only made if the area of the myocardium in which the electrode module configured in stimulation mode is located is not in a repolarisation phase.

### BRIEF DESCRIPTION OF THE DRAWINGS

The invention will be better understood and special features will become clear after reading the following description given as a non-limitative example accompanied by the appended drawings in which:

FIG. 1 is a sectional view of a human heart,

FIG. 2 is a diagrammatic front view of a heart fitted with a pacemaker according to the invention,

FIG. 3 shows a charge and discharge circuit used to apply a stimulation to the heart,

FIG. 4 illustrates the shape of a characteristic signal at a given frequency,

FIG. 5 shows a side. view of an electrode module,

FIG. 6 shows a top view of the electrode module in FIG. 5,

FIG. 7 is a functional flowchart showing operation of the electrode module in exploration mode,

FIG. 8 is a functional flowchart showing operation of the electrode module in stimulation mode,

FIG. 9 shows a partitioning into function blocks of the ASIC electronics used in the modules.

### DETAILED PRESENTATION OF PARTICULAR EMBODIMENTS

We will apply the invention to the case of a patient suffering from heart failure due to desynchronised contraction of the ventricles, to illustrate the operating principle of the pacemaker according to the invention.

Remember that the heart is divided into four cavities; the left and right atriums, and the left and right ventricles. Propagation of the depolarisation wave starts from the sinoatrial node located in the right atrium and then propagates throughout the right atrium and then in the left atrium, thus provoking mechanical contraction of the right and then left atriums to expel blood in the ventricles. In the meantime, the wave has propagated between the ventricles and provokes contraction of the left and right ventricles, which enables ejection of blood in the arteries.

In the special case of a patient suffering from desynchronised contractions of the ventricles, only the ventricles need to be equipped with stimulation electrodes. However, the atriums may be provided with electrodes, and particularly electrodes to explore the state of the heart and possibly to control the stimulation electrodes placed on the ventricles. The electrodes can thus be slaved and the stimulations, and therefore the ventricles, can be synchronised.

The implantable pacemaker used in this application example comprises a control box, a conducting power supply cable, electrode modules with processing and/or control electronics, and a net with a regular mesh used to connect electrode modules to each other. Obviously, other configurations are possible (net with non-regular mesh, no net, etc.).

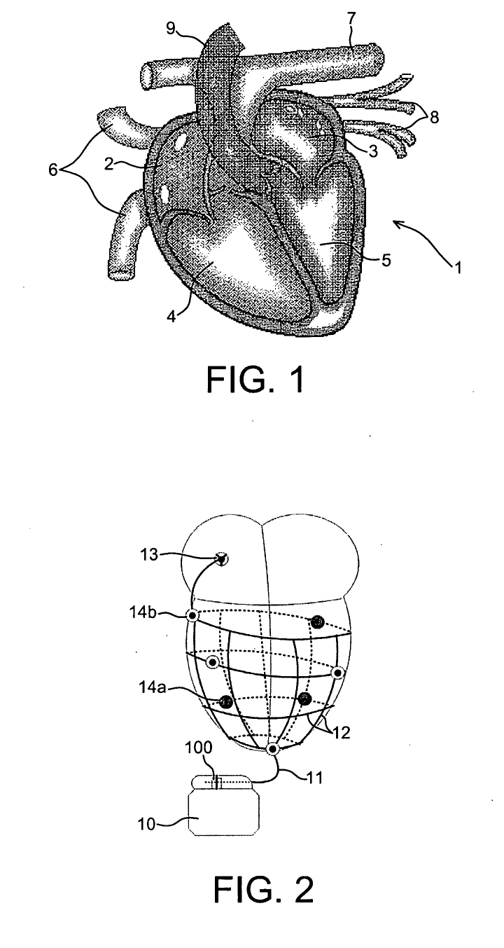

FIG. 2 includes a representation of the heart of this patient who wears a pacemaker after a surgical operation. In this example, there is an isolated electrode module 13 in exploration mode, located on the right atrium of the heart. This electrode module 13 is connected to a control box 10 that might include other functions, through a conducting power supply cable 11 that reaches the heart passing through an endocavitary vein. In this case, the power supply cable 11 is connected to the control box 10 through a screw connector 100 that is used to fix the cable 11 to the box 10. The control box 10 includes a power supply system and is located subcutaneously, in the same way as existing pacemakers. The pacemaker is powered by a 2.8 V lithium-iodine battery located in the control box 10. The control box 10 is preferably made of titanium. It will be noted that the control box 10 may consist of nothing more than a power supply system if the entire electronics is remote from the electrode modules. The power supply cable also electrically connects a multi-wire coaxial cable 12 (shown in dashed lines on the hidden side of the heart) that surrounds part of the heart ventricles and on which electrode modules are fixed. The multi-wire coaxial cable 12 could have been replaced by a net, for example with regular meshes, surrounding the ventricles and on which the electrode modules would have been placed. During the surgical operation, the surgeon places the coaxial cable fitted with the electrode modules 14a (located on the hidden side of the heart in FIG. 2) and 14b (located on the visible face of the heart) epicardially (in other words on the outside of the heart). In this example, an electrode module 13 was positioned configured in exploration mode at the right atrium, four electrode modules configured in stimulation mode on the right ventricle, and another five on the left ventricle, the electrode modules 14a, 14b located on the ventricles being connected by the coaxial cable 12. Note that FIG. 2 shows a front view of the patient's heart, which only shows a part of the electrode modules.

The coaxial cable 12 (or the net) comprising the electrode modules 14a, 14b is placed on the outside surface of the heart at the ventricles. The electrode modules shown in this figure have a round form. In FIG. 2, it can be seen that the power supply cable 11 supplies electricity to the electrode module configured in isolated exploration mode and located on the right atrium, and the coaxial cable 12 on which the electrode modules 14a, 14b in stimulation mode are located.

In this case, the coaxial cable 12 holds the different electrode modules 14a, 14b in place on the heart, supplies electricity to the electrode modules that are fixed to it and also enables the said electrode modules to communicate with each other. The electrode modules 13, 14a, 14b are positioned at judiciously chosen sites located on the surface of the heart. If the coaxial cable 12 is replaced by a net, the electrode modules 14a, 14b connected to each other by meshes of the net can be positioned on the meshes at judiciously chosen sites that may or may not be located at nodes in the mesh. Note that the net can cover all of the heart or only part of it, it may or may not be regular, and may or may not be dense. The net and all parts in contact with the heart and its surroundings are made of biocompatible materials (for example coated with silicone or titanium).

The meshes of the net are made from wires made of a conducting material. In particular, the meshes of the net are composed of several wires wound together coaxially in a helical spiral, these wires being coated with an electrically insulating material biocompatible with human tissue. For example, this electrically insulating biocompatible material may be high performance silicone. By using several wires to make the meshes of the net, the electrode modules can operate in bipole mode with electrical energy being supplied to the electrodes to stimulate or explore the myocardium through a closed electrical circuit.

The net or the coaxial cable 12 on which the electrode modules 14a, 14b are arranged, is assembled before the surgical operation. The net or the coaxial cabled fitted with the necessary electrode modules is made larger than the heart, to facilitate positioning on the heart. The meshes of the net can then be tightened after the net has been put into position. By varying the tightness of the meshes around the heart, the net 12 can also be made to apply contention to the heart (for example to treat a case of ventricular hypertrophy).

If the net's sole function is contention of the heart, the electrode modules must be connected to conducting cables through which they receive electrical energy. In this case, each cable is fitted with a coaxial connector at its ends, to provide an easy connection to electrode modules. For example, the connection could be made by screwing. In FIG. 2, the device comprises only one cable acting as the power supply cable 11. In this case, it is the net which supplies electrical energy to the electrode modules.

In this example embodiment, the electrode module in exploration mode provides the pacemaker with means of regulating the stimulation rate obtained by the electrode modules in stimulation mode. This example consists of a multi-site pacemaker with a net of slaved electrodes, in other words the stimulator comprises several stimulation sites arranged on a net, the stimulation sites being slaved to the results output by the electrode module located on the right atrium.

Electrode modules according to the invention are composed of a processing and/or control electronics, and are used to process signals from the heart and possibly from the net and to apply control over triggering of a stimulation. This processing and/or control electronics is composed of an application specific integrated circuit (ASIC) that allows to carry out this processing and/or this control. This application specific integrated circuit (ASIC) is a SoC (System on Chip), designed using SIP (Semiconductor Intellectual Property) or IP (Intellectual Property) blocks. It provides an electrical power supply regulation that enables operation of this ASIC.

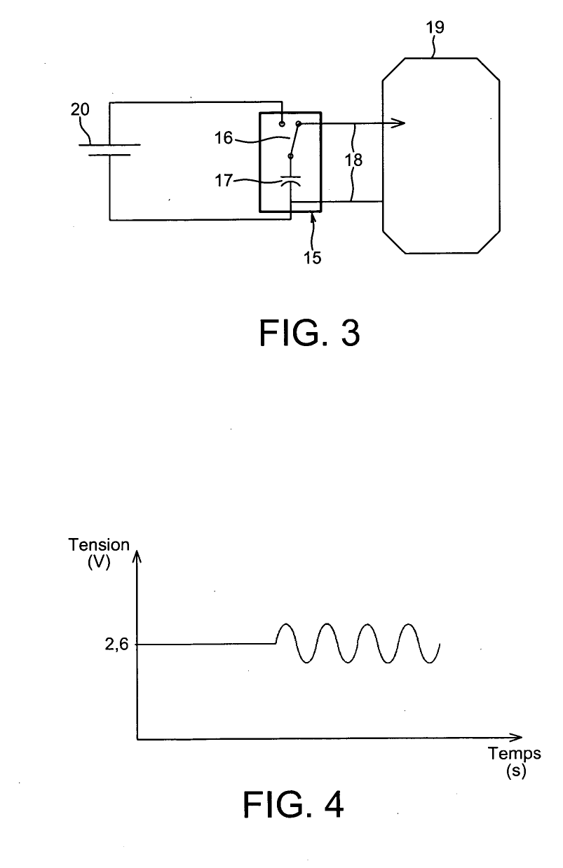

Stimulation may be achieved due to the presence of a capacitor that can be actuated by a switch controlled by each electrode module with a stimulation function. FIG. 3 diagrammatically shows an electrode module capable of operating in stimulation mode 15 that is capable of controlling actuation of a switch and thus outputting a stimulation due to the presence of a capacitor 17 (in this case, the switch and the capacitor are located in the electrode module, but they may also be remote) through the electrode 18 (in two parts) located in an area of the myocardium 19. The capacitor 17 is recharged by a 2.8 V battery 20. After charging, the capacitor 17 outputs a sufficiently large pulse to provoke a depolarisation wave by stimulation. The capacitor may be charged directly by the power supply of the control box 10. The stimulation is then triggered by the electrode module due to its electronic circuit (ASIC) when necessary.

The state of the heart (depolarisation, repolarisation) is explored or picked up using filters located in the electrode modules acting in exploration to capture signals characteristic of depolarisation (frequency from 10 to 120 Hz and voltage from 1 to 20 mV) and repolarisation (frequency less than 5 Hz and voltage less than 7 mV). In this case, the ASIC performs this information processing. The ASIC located in the electrode modules in exploration mode also slaves the stimulation electrode modules by picking up and coding a characteristic signal in voltage (FIG. 4) so as to send it to electrode modules in stimulation mode, which will or will not stimulate the heart depending on the received voltage. In this case, the meshes of the net are used to exchange this signal between electrode modules in exploration mode and the electrode modules in stimulation mode. This signal is superposed on the power supply voltage that already circulates in the meshes of the net. FIG. 4 shows an example of the characteristic signal shape at a given frequency superposed on the 2.8 V dc power supply voltage. The characteristic signal is encoded at a given frequency with a determined amplitude that is generally equal to about 0.25 V. The characteristic signal transports information on a frequency, this frequency reflecting the state of activity of the patient at a given moment. We can identify three heart rates for each patient, namely the rest rate (less then 70 beats per minute), the active rate (between 71 and 140 beats per minute) and the super-active rate (more than 140 beats per minute). This information about the heart rate is then frequency encoded on the characteristic signal at 1 kHz, 10 kHz and 100 kHz respectively.

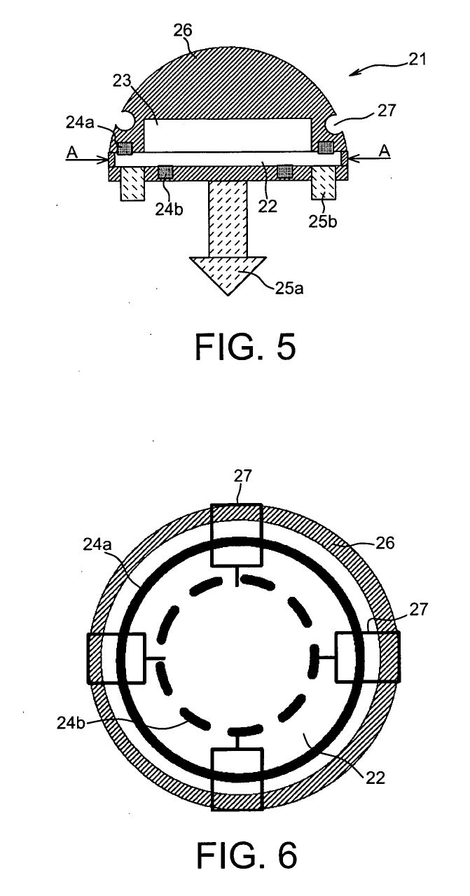

The structure of the electrode modules in exploration mode and/or stimulation mode is illustrated in FIGS. 5 et 6. In FIG. 5, it can be seen that each electrode module 21 in exploration mode and/or in stimulation mode comprises a printed circuit 22 on which an electronic circuit (ASIC) 23 is mounted that controls a stimulation and/or encodes a signal. The printed circuit 22 comprises a conducting track 24a, 24b on its upper face and on its lower face, connecting the printed circuit 22 to a pole of the battery. Thus, each conducting track 24a,24b is connected to a different pole of the battery. An electrode 25a,25b is used to electrically connect the electrode module 21 to the heart. The electrode 25a,25b is composed of two armatures made of a porous material, for example carbon. One of these armatures is located in a central position 25a with respect to the printed circuit 22 and has one end in the form of a tip or an arrow. This central electrode armature 25a may be 3 mm long. The other armature 25b is located at the periphery of the printed circuit and is used to close the electrical circuit on the area of the heart on which it is implanted in order to explore it or to stimulate it. When the net and the electrode module are being positioned on the heart, the pointed armature 25a is forced to penetrate into the myocardium at the required position. All elements making up the electrode module except for the electrode 25a, 25b are coated with an insulating material (for example silicone) biocompatible with the heart and its surroundings. In the example illustrated in FIG. 5, the coated electrode module has a rounded face convex on the top and a round smooth face. on the opposite face showing the armatures of the electrode 25a, 25b. In its convex part, the silicone coating 26 in this example is provided with symmetric notches that the surgeon uses to grip the electrode module.

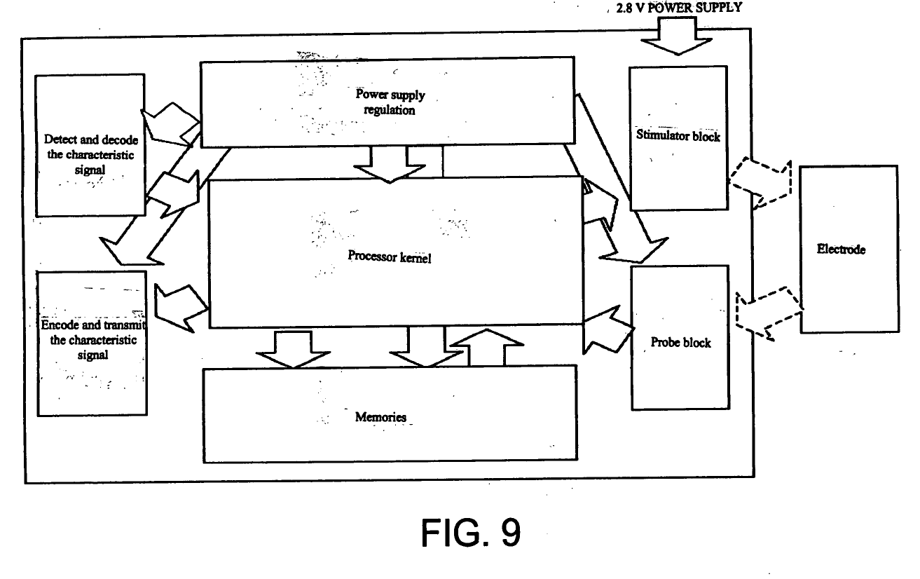

The electrode module shown in a front view in FIG. 5 is shown in a top view along section A-A in FIG. 6. To improve clarity, the conducting tracks of the printed circuit of the upper face 24a and the lower face 24b are shown with the printed circuit 22 and the four connectors 27 used to connect the electrode modules to each other through coaxial conducting wires of the net meshes and to connect them to the conducting tracks 24a, 24b on the printed circuit. The conducting tracks enables to connect the ASIC containing the power supply regulation, the capacitor to stimulate and/or the exploration block to the electrode (see FIG. 9). The ASIC is connected to the module power supply tracks. Energy regulation provides a good power supply level to the various blocks (main processor, memories, sensor, stimulator, detection and decoding of the characteristic signal, etc.). It charges the capacitor for stimulation.

The stimulator block outputs the electrical pulse to stimulate the area of the heart on which the module is implanted. This stimulus is triggered by the main processor by the switch control (see FIG. 3).

The exploration block provides information about the state of the heart (depolarisation repolarisation, or no electrical activity; constant voltage) on the area on which the module is implanted. This information is transmitted to the main processor.

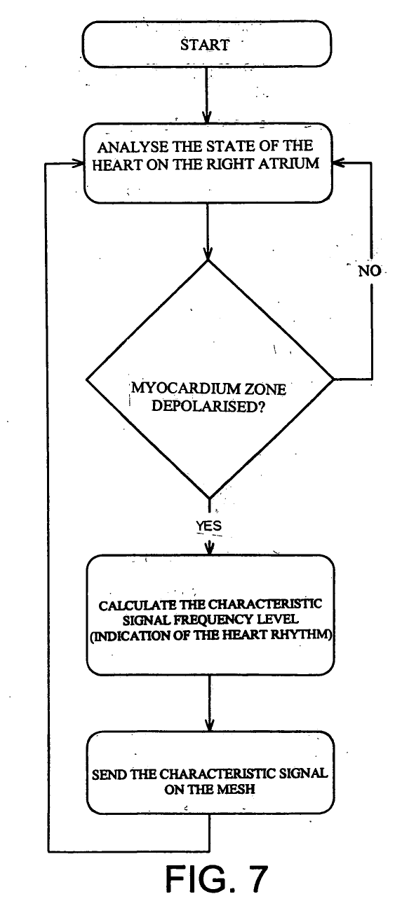

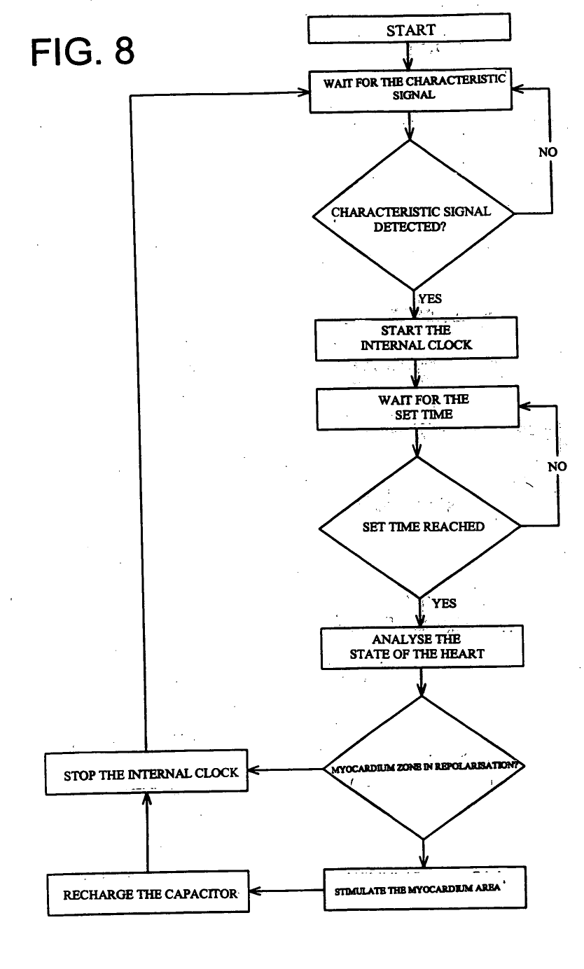

The main processor controls and communicates with all functions of the module (stimulation, probe, decoding, memories, etc.) depending on the onboard software and in accordance with the flowcharts (FIGS. 7 and 8). This software is stored on the memory blocks.

We will now describe the actual operation of the different electrode modules in more detail.

In FIG. 2, the electrode module 13 located on the right atrium is configured in exploration mode. It slaves electrode modules in stimulation mode 14a,14b connected through the net by detecting the depolarisation wave from the right atrium and by synchronising the stimulations. Since the right atrium functions normally, the electrode module 13 in exploration mode perceives the patient's normal heartbeats. It determines the heart rate by calculating the time that elapses between two successive depolarisations and it transmits this information to electrode modules configured in stimulation mode. By default, the stimulator will function at the rest rate, and therefore by default the characteristic signal will be encoded at a frequency of 1 kHz.

When the electrode module in exploration mode detects depolarisation of the area of the myocardium in which it is implanted, it sends a characteristic voltage signal through the meshes of the net (see FIG. 4) to the other electrode modules positioned on the ventricles, coding the said signal at a frequency of 1 kHz, 10 kHz or 100 kHz, depending on the calculated heart rate. It thus informs the other electrode modules about the beginning of a new coordinated depolarisation, contraction and repolarisation cycle of the myocardium, at a given frequency. All these steps performed by the electrode module in exploration mode are shown diagrammatically in FIG. 7. The electrode module in exploration mode analyses the state of the heart by picking up electrical signals sent by the heart. It then calculates the frequency level of the received electrical signal if the area of the myocardium in which it is located is in the depolarisation state, it encodes this electrical signal in frequency and then sends it to the electrode modules in stimulation mode on the net.

The electrode modules located on the ventricles are configured in stimulation mode. Their functional steps in stimulation mode are illustrated in FIG. 8. As can be seen in this figure, the electrode modules in stimulation mode are waiting for the signal characteristic of the beginning of the frequency encoded depolarisation cycle. When this signal arrives in an electrode module, it triggers a counter or an internal clock. Each electrode module is programmed with a set time specific to it. This set time is determined starting from the position of the electrode module on the myocardium and the frequency encoding (in other words, the lifestyle of the equipped patient) of the characteristic signal. This information about the lifestyle is provided by the electrode module in exploration mode positioned on the right atrium. As we have already seen, the heart frequency is encoded using three levels, on the frequency of the characteristic signal to define the patient's lifestyle—rest rate, active rate or <<super active>> rate. There is a determined set time for each lifestyle and for a given position of the electrode module in stimulation mode on the ventricles. Once the set time has been reached, the electrode module analyses the state of the heart. If the area of the myocardium in which the electrode module is located is in repolarisation, the internal clock is stopped and the electrode module returns into a state of waiting for a characteristic signal from the electrode module in exploration mode. If the area of the myocardium is not in a repolarisation state at the end of the set time, the electrode module then triggers a stimulation of the area of the myocardium concerned, the capacitor is recharged and the internal clock is stopped. Thus, for each lifestyle, depolarisation of the myocardium of the ventricles is coordinated so that they can be resynchronised and to correct the pathology of the patient.

Each electrode module may have a determined function of exploration or stimulation, but it may also be equipped with processing electronics and control electronics so that it can exercise these two functions depending on the need. In this case, the electrode module will be configured in exploration mode or in stimulation mode, depending on the case. A communication means may be placed in the control box 10 so that each electrode module in the stimulator can be configured by remote transmission and so that the electrode modules can be configured remotely at any time.

In the example described above, the electrode modules located on the ventricles and on the right atrium are composed of the same elements. The electrode modules are composed of an electrode and processing and control electronics. They process all signals from the heart or the mesh and exert control over triggering a stimulation. Electrode modules positioned on the ventricles observe the area on which they are positioned, through the electrode installed in them. When the counter reaches the set time for one of these electrode modules, it only sends a stimulation if the area of the heart on which it is located is not in a repolarisation state. In this case, it stimulates this area through its electrode by sending an electrical discharge to it, for example through a capacitor. The stimulation of myocardium cells must be greater than a stimulation threshold known using the Lapicque curve, if it is to generate a contraction. The stimulation corresponds to a value of about 2 volts for a maximum duration of half a millisecond. This stimulation provokes depolarisation of the cells in the myocardium in the area in which the electrode is located. Once the stimulation has been made, the capacitor is recharged and the internal clock counter is reset to zero while waiting for a new characteristic signal.

In this example, the net connecting the electrode modules together performs the functions of physically holding the electrodes on the heart, of communication between electrodes and of distribution of energy for the electrodes. The net thus enables coordinated and efficient operation of the electrode modules.

Advantageously, when there are several electrode modules, it is preferred to use the net for energy distribution. This avoids the need to connect each electrode module to the control box through a power supply cable and thus eliminates one cause of increased volume.

In this example, the pacemaker according to the invention can resynchronise contractions between the atriums and the ventricles, but it could also resynchronise atriums with each other or ventricles with each other. It can also be used to control the contraction dynamics of one or both atriums, and/or one or both ventricles. The presence of the net fitted with electrode modules enables stimulation of the heart as much as but no more than necessary, at the right time and at the right location. In this case, the net connects electrode modules together, provides mechanical support for the electrode modules on the heart, enables communication between electrode modules and distributes energy for the electrodes. The net provides a coordinated and efficient operation.

The pacemaker according to the invention has many advantages.

Delocalising at least part of the electronics contained in the box of a pacemaker according to prior art directly on the heart releases space so that smaller boxes can be made or a larger battery can be installed. This increases battery life and therefore extends the period between two battery changes. Furthermore, the stimulator according to the invention is the same size as or smaller than stimulators according to prior art. Therefore, patients suffering from heart failure can be equipped with them without problems. It can be used instead of a traditional pacemaker in most cases.

At the present time, the configuration of a pacemaker is such that the electrode cable itself is just a simple conductor connecting the electrode and the control box, the power supply system (battery) and the control electronics being contained in the control box that is replaced regularly (about every four years) due to battery wear. With this sort of configuration, an electrode cable corresponds to a stimulation point in the case in which the cable is connected to a single electrode, or to several stimulation points at the same time t if the cable is provided with branch connections at one of its ends and connects several electrodes. The electrode cable can also connect an electrode (probe) used as an observation and listening point for electrical signals passing through the heart at a precise point and used to coordinate stimulations made through one or more specific stimulation electrodes. Bringing all or some of the onboard electronics now located in the stimulator box to the actual stimulation point on the heart, raises the anatomophysical limit due to the increase in the number of stimulations, or the power supply limit for a pacemaker and positioning limits with branch connections.

Miniaturising the electronics located on the heart or on the mesh if there is one, reduces the electrical consumption and increases the calculation or processing capacity of the electronics. The most recent microtechnological developments (ASIC, FPGA, MENS, etc.) can be used to miniaturise the electronics. Miniaturisation may consist of designing a SoC (system on chip) using microtechnology, and possibly in the future using nanotechnology.

Moreover, by miniaturising the electrodes, their electrical resistance becomes lower than it was with pacemakers according to prior art (more than 30 cm long) . This means lower electrical consumption and a lower electrical resistance of these electrodes, for exactly the same energy and electrical stimulation power. The effect of reducing the electrical consumption of the stimulator is to extend the working life of the battery.

One of the advantages of using a means, for example a net, of connecting electrode modules together, is that electrode modules located on this net can be slaved and their operation can be made dependent on the results of another electrode module located outside the net and used for exploration.

Moreover, in using a net, there is no need to place electrodes precisely on the heart. Electrode modules may be placed on part or all of the heart using a regular or irregular pattern.

Another advantage of the net is that it can also act as a contention net to counter hypertrophy of the ventricles due to heart failure.

Moreover, robustness problems can arise with the-technology used for existing pacemakers. It is very difficult to protect these systems against sources of external aggression or interference such as electromagnetic fields or particle fields, and manufacturers rarely provide this protection. With existing technologies, if an electrode, the control box or the battery become defective, the entire pacemaker system becomes defective and causes serious consequences for the patient. Electrode modules in the pacemaker according to the invention can operate independently. If one electrode module is damaged, the other electrode modules can continue to function.

With the pacemaker according to the invention, it becomes possible to stimulate many sites in the pericardium or the endocavitary of the heart. With this stimulator, it is possible to have a much larger number of stimulation points than with existing pacemakers that are limited to the use of a maximum of three electrode cables due to their size.

This invention increases the robustness and operating reliability of the pacemaker. By arranging miniature electrodes on a mesh, we eliminate the limit on the number of electrodes for anatomophysiological reasons, or the electrical resistance of electric cables of electrodes with branch connections that are difficult to power using standard battery technologies. We can then have a very large number of stimulation points, more than is possible with the existing stimulators technology. If more stimulation points are used, stimulation points and power supply paths can be made redundant due to the presence of the net. This redundancy of stimulation points also compensates for the pragmatic approach used by the practitioner in correctly positioning the electrodes during the surgical operation.

### BIBLIOGRAPHY

[1] US 2000/6067470 A1; <<System and method for multiple site biphasic stimulation to revert ventricular arrhythmias>>
[2] US 2002/0082647 A1; <<Cardiac disease treatment and device>>.

1. Implantable pacemaker comprising at least one conducting cable (11) electrically connecting a control box (10) to at least one electrode (18) placed at a point in the patient's heart, the control box (10) comprising a power supply system and an electronic system, characterised in that at least one part of the electronic system is remote from the control box (10) and is located at the at least one electrode (18) to form an electrode module (13, 14a, 14b, 15, 21), the electrode module being placed on the surface of the heart.

2. Implantable pacemaker according to claim 1, characterised in that at least part of the power supply system is remote from the control box (10) and is located at at least one electrode module (13, 14a, 14b, 15, 21).

3. Implantable pacemaker according to claim 1, characterised in that the part of the remote electronic system is a processing and/or control electronics, this electronics enabling the electrode module (13, 14a, 14b, 15, 21) to explore and/or stimulate the heart.

4. Implantable pacemaker according to claim 1, characterised in that the processing electronics enabling the electrode module (13, 21) to explore the heart comprises at least one probe.

5. Implantable pacemaker according to claim 3, characterised in that the processing electronics enabling the electrode module (13, 21) to explore the heart comprises a means capable of selecting signals of determined frequency.

6. Implantable pacemaker according to claim 1, characterised in that the means capable of selecting signals of determined frequency is a filter.

7. Implantable pacemaker according to claim 3, characterised in that the control electronics enabling the electrode module (14a, 14b, 15, 21) to stimulate the heart comprises a means capable of outputting an electrical stimulation to the heart.

8. Implantable pacemaker according to claim 1, characterised in that the said means capable of outputting an electrical stimulation to the heart is a capacitor (17).

9. Implantable pacemaker according to claim 1, characterised in that the part of the remote electronic system is a system on chip.

10. Implantable pacemaker according to any one of the claim 1, characterised in that it comprises at least two electrode modules (14a, 14b) connected to each other by a means that surrounds at least part of the heart.

11. Implantable pacemaker according to the previous claim 1, characterised in that the means that surrounds all or part of the heart is used for the mechanical support of the at least two electrode modules (14a, 14b) on the at least part of the heart.

12. Implantable pacemaker according to claim 10, characterised in that the means is a means used for contention of the part of the heart surrounded by the said means.

13. Implantable pacemaker according to claim 10, characterised in that the means is a means used for electrical communication between the electrode modules (14a, 14b).

14. Implantable pacemaker according to claim 10, characterised in that the said means is a means used for energy distribution between the electrode modules (14a, 14b).

15. Implantable pacemaker according to claim 14, characterised in that the said means or an electrode module (14a, 14b) located on the said means is connected to the control box (10) by a conducting cable.

16. Implantable pacemaker according to claim 13, characterised in that the said means is a multi-wire coaxial cable.

17. Implantable pacemaker according to claim 10 characterised in that the means is a net (12) comprising a mesh.

18. Implantable pacemaker according to claim 17, characterised in that the mesh of the net (12) is made from multi-wire and coaxial helical conductors covered by an electrically insulating material biocompatible with the human tissues with which they are in contact.

19. Implantable pacemaker according to claim 1, characterised in that the electronic system also comprises a communication means enabling remote configuration of the electrode modules (13, 14a, 14b, 15, 21), the communication means being located in the control box (10) and comprising an antenna operating by remote transmission.

20. Implantable pacemaker according to claim 1, characterised in that only the antenna of the communication means is located in the control box (10), the rest of the elements making up the communication means being located on the means used for connection of the electrode modules (14a, 14b).

21. Implantable pacemaker according to claim 3, characterised in that at least one electrode module (13) is configured in exploration mode and transmits information about the state of the heart to the other electrode modules (14a, 14b, 15).

22. Implantable pacemaker according to claim 3, characterised in that at least one electrode module is configured in exploration mode and in stimulation mode.

23. Implantable pacemaker according to claim 10, characterised in that all electrode modules (13, 14a, 14b, 15, 21) are located on the means of connecting the electrode modules together.

24. Implantable pacemaker according to claim 21, characterised in that at least one electrode module (13) configured in exploration mode is not located on the means of connecting the electrode modules together, but is electrically connected to it.

25. Implantable pacemaker according to claim 15, characterised in that it comprises an electrode module (13) configured in exploration mode capable of executing the following sequence of steps:
a) analyse the electrical state of the heart at the location of the electrode module (13) configured in exploration mode,
b) when receiving an electrical signal originating from the heart, calculate the frequency of the said signal to determine whether or not the heart is in a depolarised or a repolarised state,
c) if the heart is in a depolarised state, then send a frequency encoded signal to the means of exchanging information between the electrode modules (14a, 14b, 15, 21), the frequency coding giving information about the time separating two repolarisation states of the heart.

26. Implantable pacemaker according to claim 1, characterised in that it comprises at least one electrode module (14a, 14b, 15) configured in stimulation mode and that can execute the following sequence of steps:
a) wait for a frequency encoded signal originating from an electrode module (13) in exploration mode,
b) after detection of the encoded signal, start the internal clock and wait for a determined set time,
c) analyse the electrical state of the heart at the location of the electrode module (14a, 14b, 15) in stimulation mode,
d) if the electrode module (14a, 14b, 15) configured in stimulation mode receives an electric signal from the heart with a value above a determined threshold, then the internal clock is stopped and the set time is reset to zero,
e) if the electrode module (14a, 14b, 15) in stimulation mode does not receive an electric signal from the heart, then the electrode module outputs an electrical stimulation at the position at which it is located on the heart, the internal clock is stopped and the set time is reset to zero.
